(19) United States
(12) Patent Application Publication
(10) Pub. No.: US 2005/0199742 A1
(43) Pub. Date: Sep. 15, 2005

(54) Microporous membrane air freshening device

(76) Inventor: Huib Maat, Twickenhan (GB)

Correspondence Address: Paul Grandinetti;Levy & Grandinetti, Suite 408, 1725 K Street NW, Washington, DC, 20006-1419, US

(21) Appl. No.: 10/510,341

(22) PCT Filed: Apr. 1, 2003
(86) PCT No.: PCT/GB03/01483

§ 371 Date: May 13, 2005

(30) Foreign Application Priority Data
Apr. 6, 2002 (GB) 0208028.1

(51) Int. Cl. A61L 9/04

(52) U.S. Cl. 239/44; 239/34

(57) ABSTRACT

There is described a device comprising a base with at least one volatile active housed in a reservoir, at least one wall of which comprises a wicking membrane. In particular there is described a device which is a liquid air freshener.

This invention relates to a novel device incorporating a microporous membrane.

More particularly the invention relates to a novel air freshening device or a devices for releasably storing a volatile active agent.

Conventional liquid air fresheners generally comprise a reservoir containing a fragrance solution, into which dips a wick, which is connected to an emanating surface. The perfume solution travels up the wick to the emanating surface from which the perfume evaporates and freshens the surrounding atmosphere. In many examples the wick and emanating surface are formed from the same item. The fragrance solution may be a true solution (comprising a perfume base and a solvent that is miscible with the perfume base) or a colloidal solution (comprising perfume base, water, a surfactant which solubilises the perfume in the water, and optionally solvents).

One disadvantage of conventional liquid air fresheners is that the fragrance solution can leak from the packaging should the device be accidentally knocked over. This imposes certain limitations with regards how the product is used, and the types of design forms that the product can take.

More recently air freshening devices incorporating hydrophobic polymeric membranes have been developed. U.S. Pat. No. 4,145,001, to American Can Co. describes a volatile deodoriser sandwiched between the layers of a laminate material.

International Patent Application No. WO 98/23304, to Burgopack Stampa describes a container for volatile substances comprising a plurality (five) of outer impermeable plies, detachable from a permeable ply.

U.S. Pat. No. 5,804,264 to American National Can Co. describes a multilayer permeable membrane for use in air fresheners comprising low density polyethylenes.

Such devices comprise a fragrance composition contained within a sealed reservoir. At least one wall of the reservoir comprises a membrane, for example, which is constructed from a material that is permeable to perfume vapours. However, such devices suffer from the disadvantage that the water is unable to migrate through the membrane and thus the liquid, or gel, compositions used in such devices are essentially anhydrous. The compositions generally comprise a perfume base and, optionally, a volatile solvent and, optionally, a small amount of a thickening agent, such as, a fumed silica or ethyl cellulose. The compositions, being essentially free of water, tend to be relatively expensive.

Thus there has long been a need for a device which comprises a water based system but which does not suffer from the disadvantages of, inter alia, leaking, as hereinbefore described.

Teslin® (origin: PPG Industries) is described in U.S. Pat. No. 4,861,644 and comprises a single layer, highly filled, microporous film and is conventionally used as a synthetic printing sheet. Teslin® is essentially a matrix of linear ultrahigh molecular weight polyolefin a very large proportion of finely divided particulate siliceous filler, and a high void content microporous material.

More specifically, although different grades of Teslin® are available, Teslin® generally comprises a polyolefin which contains approximately 60% w/w non-abrasive filler. The structure is approximately 65% v/v air. Teslin® can be fusion bonded by impulse sealing to various thermoplastics and certain thermosets.

Teslin® is generally known to be useful as a material for, e.g. label printing.

We have now surprisingly discovered that certain known microporous materials, such as Teslin®, can be used to construct a membrane for use in, for example, an aqueous based liquid air freshening device. Such materials are especially advantageous in that they are capable of simultaneously acting as both a microporous membrane and a wick. Moreover, materials, such as Teslin® possess the ability to wick along the plane of the membrane.

Thus, according to a first aspect of the invention we provide a device comprising a base with at least one volatile agent active housed in a reservoir, at least one wall of which comprises a wicking membrane.

More particularly we provide a device according to claim 1 characterised in that the device is a liquid air freshener which comprises a perfumed air freshener base housed in a reservoir, at least one wall of which comprises a wicking membrane. Preferentially, the perfumed air freshener base is an aqueous system.

By the term wicking membrane we especially mean a membrane which is capable of performing a wicking action in the plane of the membrane.

It will be understood that the device of the invention may suitably be used for air freshening or other similar functions. Thus, the volatile active agent may be selected from an insecticide, an insect repellent, an insect attractant, a fragrance, a deodorising agent and an anti-bacterial agent, or any combination thereof

Thus according to a preferred feature of the invention we provide a liquid air freshener which comprise an aqueous perfumed air freshener base housed in a reservoir, at least one wall of which comprises a microporous membrane which membrane comprises a matrix of linear ultrahigh molecular weight polyolefin. a very large proportion of finely divided particulate siliceous filler, and a high void content.

In a preferred embodiment of the invention the microporous membrane may comprise Teslin®.

The thickness of the membrane may vary and may be for example, from 0.05 to 2.0 mm thick, preferably from 0.7 to 1.8 mm, more preferably from 0.8 to 1.4 mm, e.g. 1.0 mm thick.

The remaining walls of the reservoir may comprise any conventionally known material, for example, a plastics material. Any conventionally known plastic material may be used, such as, for example, of polyethylenes, polypropylene, polymethyl methacrylate, ABS, polystyrene, rigid PVC, PMMA, Polyester and polycarbonate. A preferred material is a polyolefin, e.g. polypropylene.

Alternatively, the plastics material may comprise a laminate material. Such a laminate may comprise any of the aforementioned materials. In a further embodiment the laminate may comprise one or more of the aforementioned materials bonded to a more robust material such as glass, ceramic, metal, wood, stone, etc.

Preferentially, the reservoir surface, or at least an edge of the surface, may comprise a weldable material, thus facilitating welding to the membrane. Examples of such weldable materials include, but shall not be limited to polyethylenes, polypropylene, polyethylene or polypropylene laminates, such as PET/PE laminates, PET/PP laminates and Barex/PE laminates.

The Teslin® may be fusion bonded to, for example, a flat panel samples of polyethylenes, polypropylene, polymethyl methacrylate, ABS, polystyrene, rigid PVC, polycarbonate, Barex, PET and Plexiglas.

Alternatively, the Teslin may be attached by other means, such as but not limited to a suitable adhesive, or it may be compressed between the container and a restraining collar through for example but not limited to a screw or clipping mechanism in the collar and/or container.

The perfume solution may be a conventional aqueous composition comprising water, at least one surfactant, a perfume base and optionally one or more solvents.

The perfume solution may generally comprise between 0.01% to 100% w/w of perfume, 0.01% to 99.99% w/w of water, 0.01% to 40% w/w of one or more suitable surfactants and optionally 0.01% to 99.99% w/w of one or more suitable solvents. Usually the concentration of perfume is between 8 and 20% by weight, often about 10% by weight.

Alternatively it may be an aqueous, surfactant free composition such as those described in U.S. Pat. No. 4,663,081 or U.S. Pat. No. 6,180,595 incorporated herein by reference.

Alternatively, it may be a composition consisting of 100% fragrance or of fragrance and solvent only.

The microporous structure of the Teslin® absorbs the perfume solution, which can then evaporate to the surrounding atmosphere. Further, the microporous properties of Teslin® facilitate wicking inside the membrane ensuring that the evaporative surface area remains saturated with perfume solution throughout the life of the product thus guaranteeing consistent fragrance intensity over the lifetime of the product. Furthermore, even when used in an inverted orientation with the Teslin® downwards, the perfume solution does not drip or leak from the device.

In yet a further embodiment of the invention the reservoir may be provided with a wicking means. The wicking means may comprise a conventional wicking member, e.g. a central wick which abuts the membrane surface. Alternatively, the wicking means may comprise one or more capillary grooves in the wall of the reservoir. In a yet further alternative, the wicking means may comprise an inner liner in the reservoir, which may be a hard sleeve or a self expanding film, for example, the inner sleeve/film may rest on the floor of the reservoir and abut the inner surface of the membrane or the inner sleeve/film may have its ends bent inwards to have a wider surface that rests on the floor of the reservoir and abuts against the inner surface of the membrane. This would improve the wicking action of the sleeve/film. The inner sleeve/film may be continuous or discontinuous, whereby the ends of the discontinuous sleeve/film may overlap or be short of each other.

To prevent the fragrance solution from evaporation prior to activation by the consumer, the Teslin® membrane may be covered with a barrier material, such as but not limited to PE, PA, PP, PET, Barex, Nylon, Aluminium or a laminate comprising but not limited to one or more of these materials. Such barrier material may be weakly bonded to the Teslin® material through for example a fusion bond or weld, a suitable adhesive or a special release layer such as described in the previously described patents: U.S. Pat. No. 4,145,001, to American Can Co; International Patent No. WO 98/23304, to Burgopack Stampa; and U.S. Pat. No. 5,804,264 to American National Can Co. Alternatively, the barrier material may be weakly bonded to the Teslin® material at certain points only, for example the edge.

According to a further aspect of the invention we provide a lavatory cleanser/deodoriser comprising a device as hereinbefore described.

We further provide an insecticide, insect attractant, insect repellent, deodoriser, germ killer comprising a device as hereinbefore described.

In an additional aspect of the invention we provide a device comprising a base with at least one non-volatile active housed in a reservoir, at least one wall of which comprises a wicking membrane.

In such a device at least one of the non-volatile materials is selected from but not limited to a surfactant, a colouring agent, an anti-bacterial agent, an acid, a bleach, a solvent and a thickening agent.

We further provide a method of manufacturing a device as hereinbefore described which comprises fusion bonding, gluing or compressing a membrane to a plastics reservoir which reservoir is adapted to house a device.

In a preferred method of the invention the microporous membrane comprises Teslin®.

We also provide the use of a microporous membrane comprising a matrix of linear ultrahigh molecular weight polyolefin, a very large proportion of finely divided particulate siliceous filler, and a high void content in the manufacture of a device as hereinbefore described.

In a preferred aspect of the invention we provide the use of the invention wherein the membrane comprises Teslin®.

The invention will now be described by way of example only and with reference to the accompanying drawings.

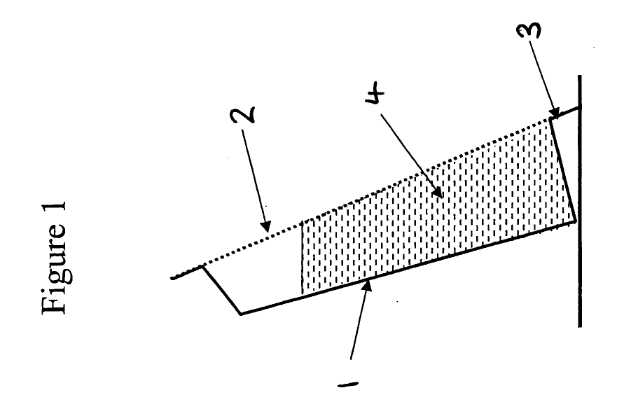

FIG. 1 shows a diagram of a liquid air freshening device that comprises a moulded polypropylene reservoir, to which is fusion bonded a Teslin® microporous film;

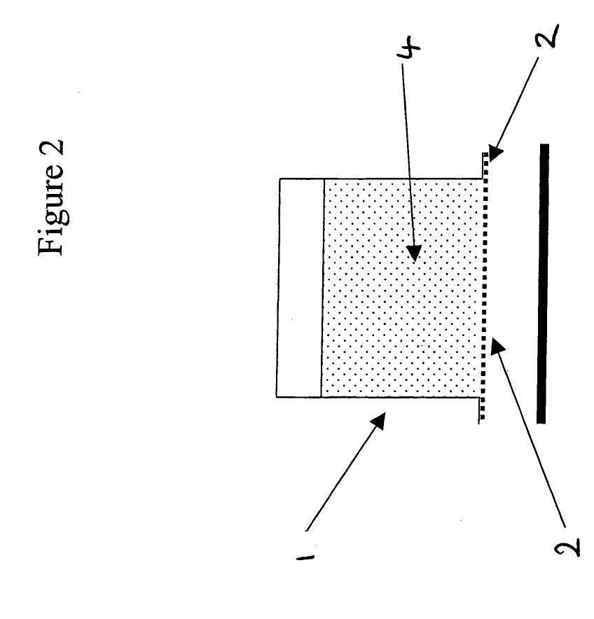

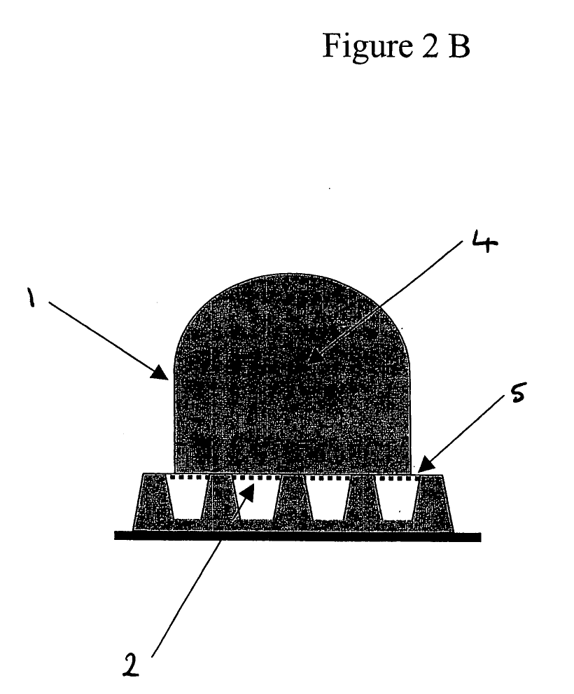

FIG. 2 is a schematic representation of an inverted reservoir;

FIG. 2b is a schematic representation of a Teslin® film fusion bonded to a polypropylene film;

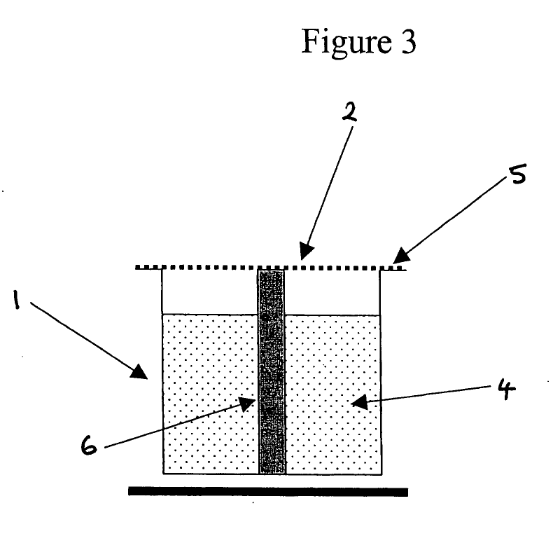

FIG. 3 is a schematic representation of a reservoir provided with a central wick;

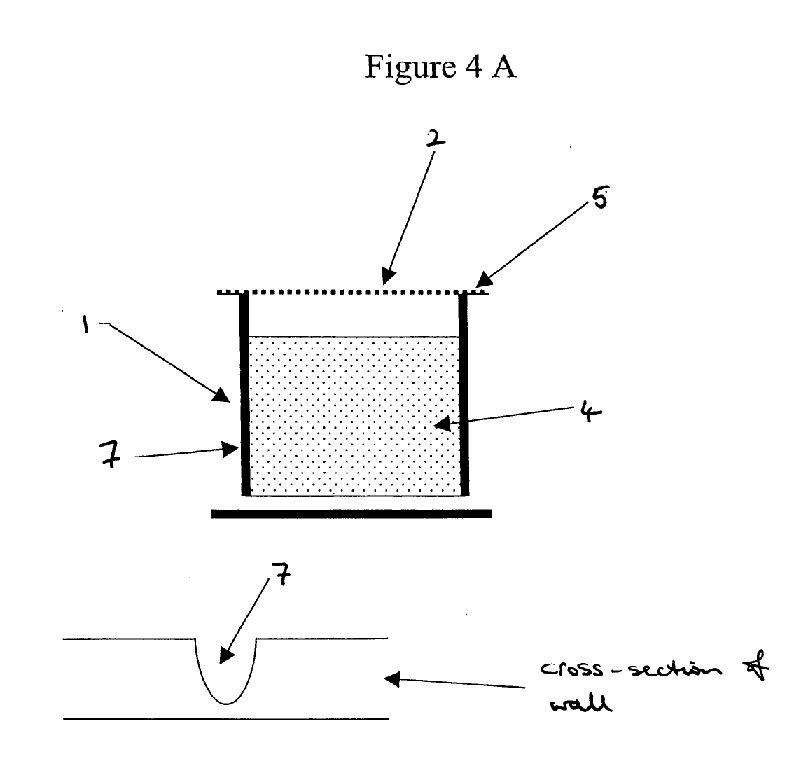

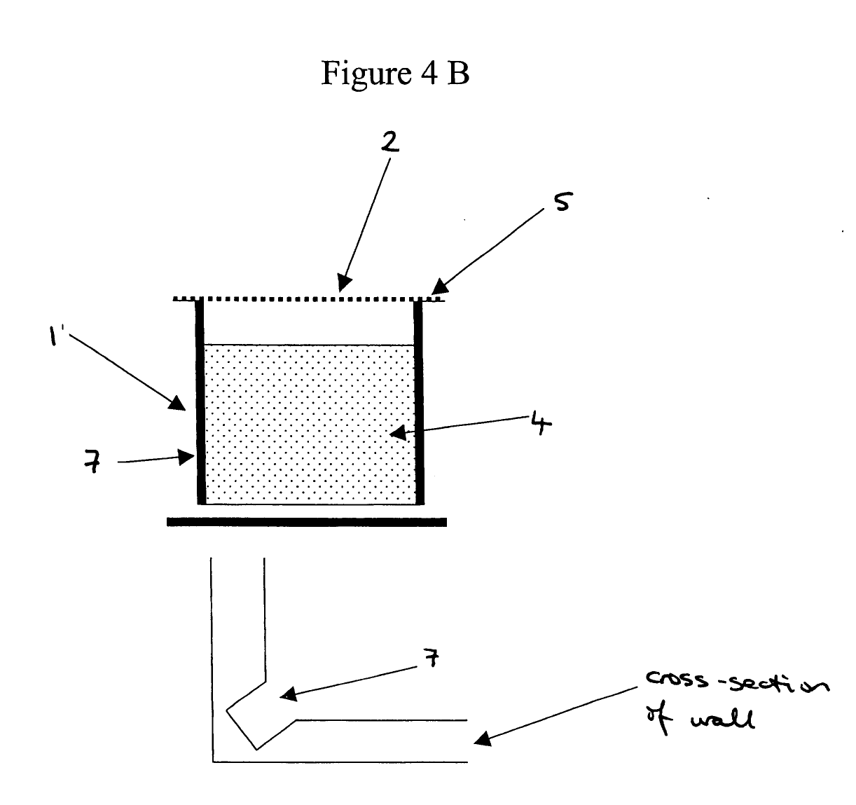

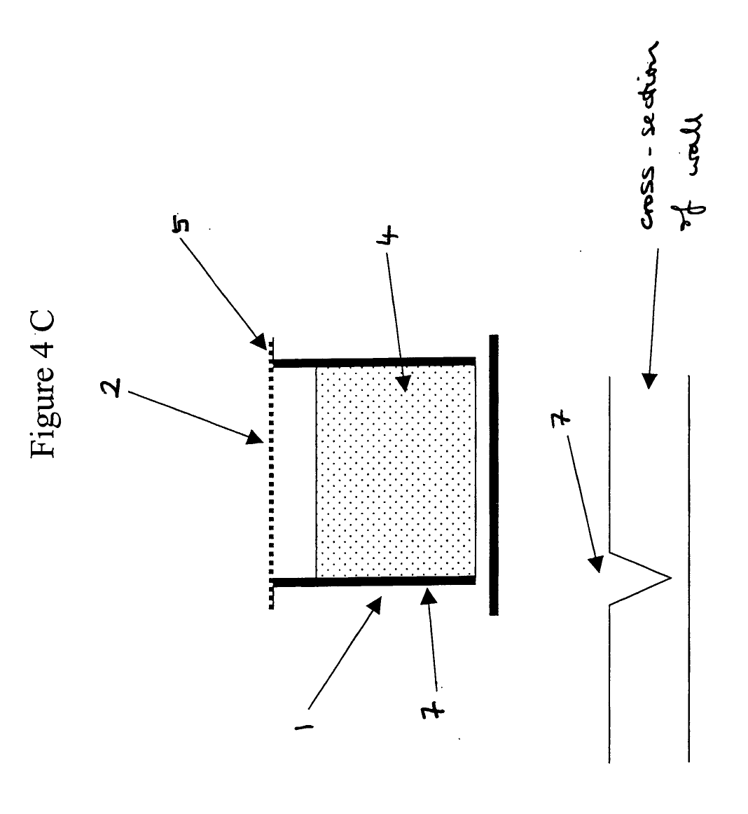

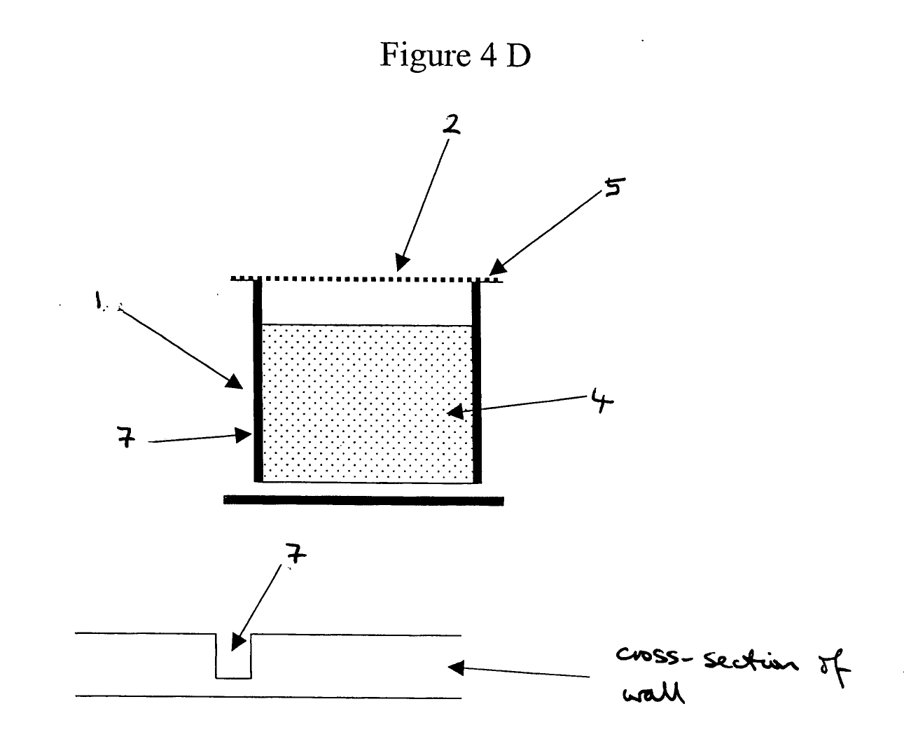

FIGS. 4a to 4d are schematic representations showing capillaried walls;

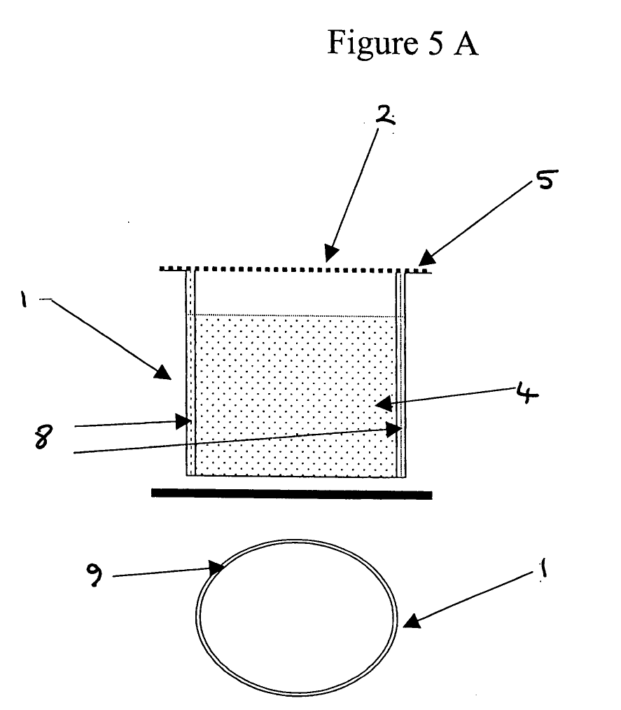

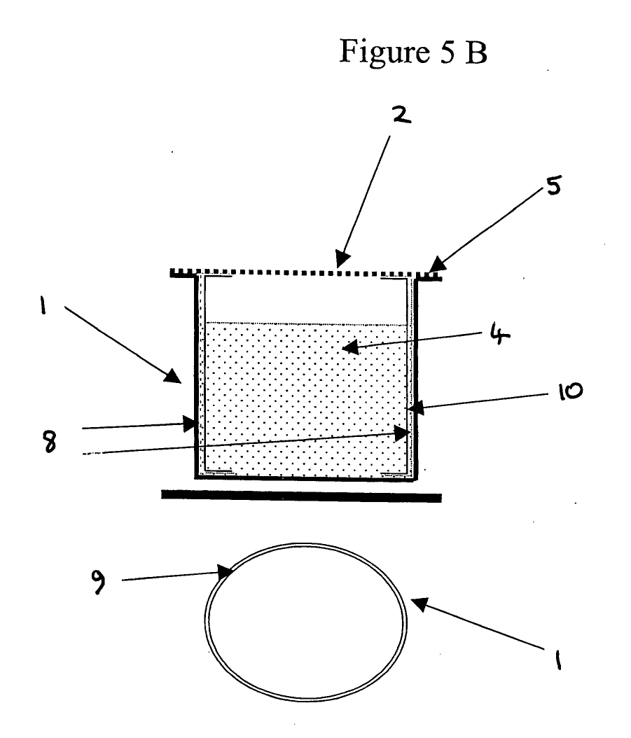

FIG. 5a to 5b are schematic representation of a reservoir provided with an inner film or sleeve;

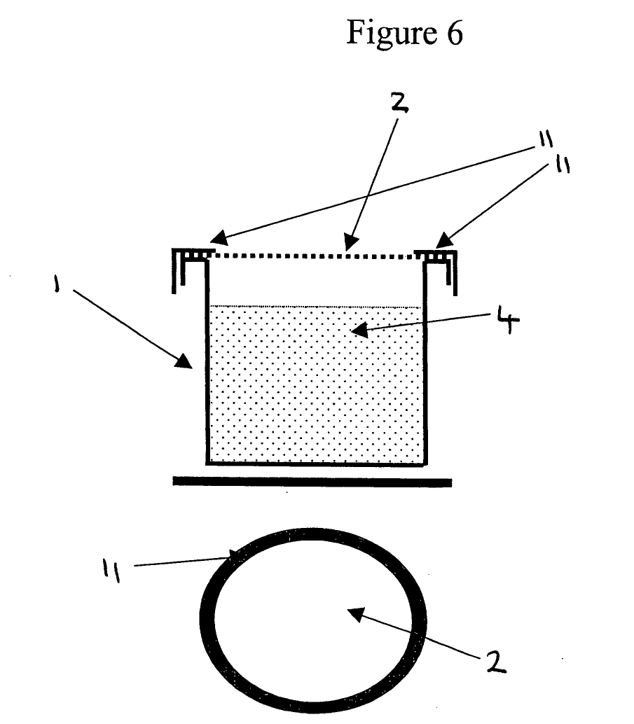

FIG. 6 is a schematic representation of a constraining collar compressing the Teslin® film to the reservoir;

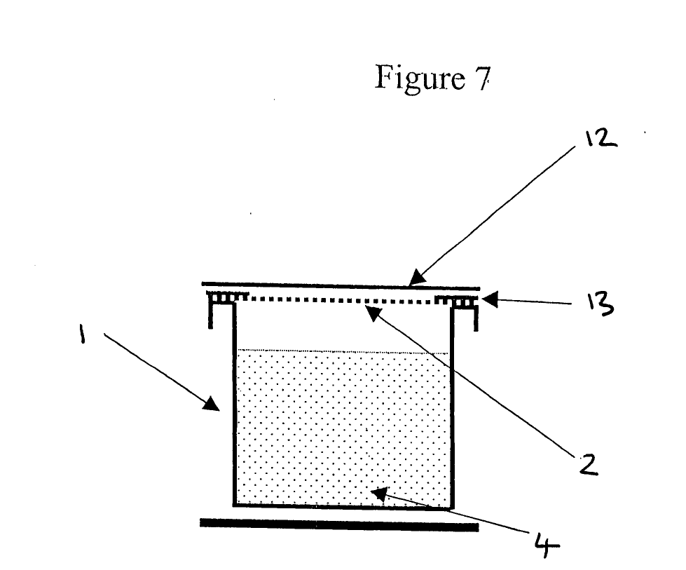

FIG. 7 is a schematic representation of a reservoir in which a barrier film is held on the Teslin® film by a weldable material; and

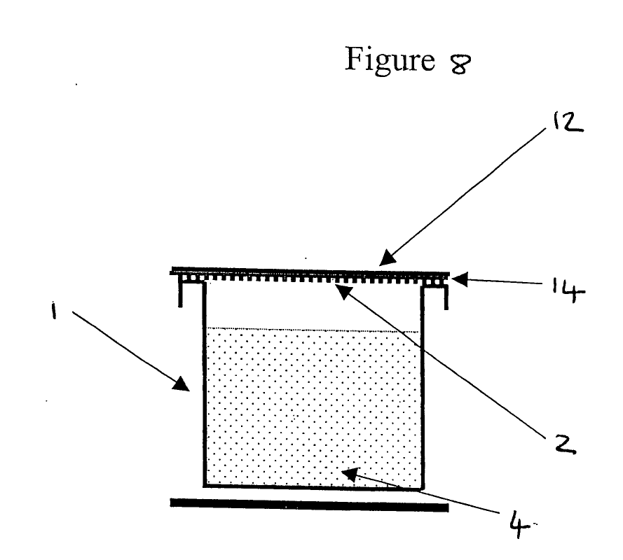

FIG. 8 is a schematic representation of a reservoir in which a barrier material is held on the Teslin® film by an adhesive or a special release layer.

Referring to FIG. 1, a plastics, e.g. polypropylene, reservoir (1) comprises one wall (2), e.g. the front wall, which is a Teslin® membrane. The membrane is fusion bonded to the walls of the reservoir. Preferentially, the base of the reservoir is provided with a lip (3) at the end of the front face, enabling the reservoir to stand vertically by resting on the lip and the rear of the base. In use, the reservoir may be filled, or partially filled, with a liquid air freshener (4).

Referring to FIGS. 2 and 2b, a reservoir may be inverted and the Teslin® film does not leak the perfume solution. The Teslin® film may be film fusion bonded (5) to the polypropylene reservoir.

Referring to FIG. 3, a reservoir may have the membrane at the top and to ensure continuous feeding of the membrane the performance of the air freshener may be improved by incorporating a central wicking member (6) into the reservoir.

Referring to FIGS. 4a to 4d, as an alternative to the central wicking member, the inner surface of the reservoir walls may be provided with a plurality of capillaries (7) which facilitate the transfer of the perfume solution to the membrane surface.

Referring to FIGS. 5a and 5b, the reservoir may be provided with an inner liner (9) in the form of a sleeve or a film. The inner liner (i.e. sleeve/film) sits on the base of the reservoir and the distal end of the liner abuts the inner membrane surface. Thus, the capillary action (8) of the liner transports the perfume solution to the inner membrane surface. Alternatively, the liner may be a sheet or film that is inserted into the outer container and forced into the shape by the outer container, thus lining the inside wall of the outer container. In this way the two ends of the sheet may overlap or be short of each other. The tips of the liner (10) may be bent inwards to improve wicking action.

Referring to FIG. 6, the reservoir may be attached to a collar (11) via a screw or clipping mechanism that compresses the Teslin® film against the reservoir, sealing the reservoir and preventing leakage at the edge.

Referring to FIGS. 7 and 8 a barrier material (12) is fixed to the Teslin® film (2) by the use of a circumferential strip of weldable material (13). In FIG. 8 the weldable material is replaced by a weakly bonded adhesive or special release layer (14).

1. A device comprising a base with at least one volatile active agent housed in a reservoir, at least one wall of which comprises a wicking membrane.

2. A device comprising a base with at least one non-volatile active housed in a reservoir, at least one wall of which comprises a wicking membrane.

3. A device according to claims 1 characterised in that the device is a liquid air freshener which comprises a perfumed air freshener base housed in a reservoir, at least one wall of which comprises a wicking membrane.

4. A device according to claim 1 characterised in that the active volatile material is selected from an insecticide, and insect repellent, an insect attractant, a fragrance, a deodorising agent and an anti-bacterial agent, or any combination thereof.

5. A device according to claim 1, characterised in that the perfumed air freshener is an aqueous perfumed air freshener.

6. A device according to claim 1 characterised in that the membrane comprises a matrix of linear ultrahigh molecular weight polyolefin, a very large proportion of finely divided particulate siliceous filler, and a high void content.

7. A device according to claim 6 characterised in that the microporous membrane comprises Teslin®.

8. A device according to claim 1 characterised in that the thickness of the membrane is from 0.05 to 2.0 mm.

9. A device according to claim 8 characterised in that the thickness of the membrane is from 0.7 to 1.8 mm.

10. A device according to claim 9 characterised in that the thickness of the membrane is from 0.7 to 1.4 mm.

11. A device according to claim 1 characterised in that the remaining walls of the reservoir comprise a plastics material.

12. A device according to claim 11 characterised in that the plastics material is selected from one or more of polyethylene, polypropylene, polymethyl methacrylate, ABS, polystyrene, rigid PVC, polycarbonate, Barex, PET and Plexiglas.

13. A device according to claim 1 characterised in that the reservoir comprises a laminate material.

14. A device according to claim 13 characterised in that the laminate comprises a plastics material bonded to a glass, ceramic, wood, stone, porcelain, etc.

15. A device according to claim 13 characterised in that the reservoir surface, or at least an edge of the surface, comprises a weldable or fusable material.

16. A device according to claim 15 characterised in that the weldable or fusable material is a polyethylene laminates.

17. A device according to claim 16 characterised in that the laminate is selected from PET/PE laminate and Barex/PE laminate.

18. A device according to claim 12 characterised in that the plastics material is polypropylene.

19. A device according to claim 1 characterised in that the membrane is fusion bonded to the reservoir.

20. A device according to claim 1 characterised in that the membrane is attached to the reservoir by other means, such as but not limited to a suitable adhesive or restraining collar.

21. A device according to claim 1 characterised in that the reservoir is provided with a wicking means.

22. A device according to claim 21 characterised in that the wicking means comprises a central wick which abuts the membrane surface.

23. A device according to claim 21 characterised in that the wicking means comprises one or more capillaries in the wall of the reservoir.

24. A device according to claim 23 characterised in that the wicking means comprises one or more capillary grooves or tubes in the wall of the reservoir.

25. A device according to claim 21 characterised in that the wicking means comprises a continuous, overlapping or interrupted inner sleeve in the reservoir.

26. A device according to claim 25 characterised in that the inner sleeve contains one or more capillaries.

27. A device according to claim 23 characterised in that the inner sleeve contains one or more capillaries, grooves or tubes.

28. A device according to claim 1 characterised in that the concentration of perfume is in the range of from 0.01% to 100% w/w.

29. A device according to claim 1 characterised in that the perfume solution comprises water, at least one surfactant, a perfume base and, optionally, one or more solvents.

30. A device according to claim 29 characterised in that the perfume solution comprises between 5 and 30% by weight of perfume.

31. A device according to claim 30 characterised in that the perfume solution comprises between 8 and 20% by weight of perfume.

32. A device according to claim 31 characterised in that the perfume solution comprises about 10% by weight of perfume.

33. A device according to claim 1 characterised in that the perfume solution is an aqueous, surfactant free composition.

34. A device according to claim 1 characterised in that the perfume solution is blend of perfume and one or more solvents.

35. (canceled)

36. A lavatory cleanser/deodoriser comprising a device according to claim 1.

37. An insecticide, insect attractant, insect repellent, deodoriser, germ killer comprising a device according to claim 1.

38. A device according to claim 2 characterised in that at least one of the non-volatile materials is selected from but not limited to a surfactant, a colouring agent, an anti-bacterial agent, an acid, a bleach, a solvent and a thickening agent.

39. A device according to claim 38 characterised in that at least one of the non-volatile materials is water soluble.

40. A lavatory cleanser/deodoriser comprising a device according to claim 2.

41. An insecticide, insect attractant, insect repellent, deodoriser, germ killer comprising a device according to claim 2.

42. A method of manufacturing a device according to claim 1 which comprises fusion bonding, gluing or compressing a membrane to a plastics reservoir which reservoir is adapted to house a device.

43. A method according to claim 42 characterised in that the microporous membrane comprises Teslin®.

44. The use of a microporous membrane comprising a matrix of linear ultrahigh molecular weight polyolefin, a very large proportion of finely divided particulate siliceous filler, and a high void content in the manufacture of a device according to claim 1.

45. The use according to claim 44 characterised in that the membrane comprises Teslin®.

46. A device according to claim 1 characterised in that the membrane is sealed with a film member.

47. A device according to claim 46 characterised in that the membrane is sealed with a film member using one or more of an adhesive, a special release layer or a thermosetting material.

48. (canceled)

49. A device substantially as described with reference to the accompanying drawings and examples.
